(19) United States
(12) Patent Application Publication
(10) Pub. No.: US 2009/0118680 A1
(43) Pub. Date: May 7, 2009

(54) Automatic gas filling consumable

(76) Inventors: Peter Goldbrunner, Irvine, CA (US); Robert J. Sanchez, JR., Oceanside, CA (US); Mark A. Hopkins, Mission Viejo, CA (US)

Correspondence Address: ALCON, IP LEGAL, TB4-8, 6201 SOUTH FREEWAY, FORT WORTH, TX, 76134, US

(21) Appl. No.: 11/935,171

(22) Filed: Nov. 5, 2007

(51) Int. Cl. A61M 5/315

(52) U.S. Cl. 604/236

(57) ABSTRACT

An improved consumable for use in a fluid/gas exchange in vitreoretinal surgery. The consumable includes a syringe having a barrel with a first opening for receiving a retinal tamponading gas, a stopper slidably disposed within and fluidly sealed to the barrel, a plug assembly, and a plunger. The stopper has a relief valve for venting air and retinal tamponading gas to atmosphere via a hollow bore in the plunger.

### FIELD OF THE INVENTION

The present invention generally pertains to vitreoretinal surgery and more particularly to consumables for helping to perform fluid exchanges typically used in such surgeries.

### DESCRIPTION OF THE RELATED ART

In a healthy human eye, the retina is physically attached to the choroid in a generally circumferential manner behind the pars plana. The vitreous humor, a transparent jelly-like material that fills the posterior segment of the eye, helps to cause the remainder of the retina to lie against, but not physically attach, to the choroid.

Sometimes a portion of the retina becomes detached from the choroid. Other times a portion of the retina may tear, allowing vitreous humor, and sometimes aqueous humor, to flow between the retina and the choroid, creating a build up of subretinal fluid. Both of these conditions result in a loss of vision.

To surgically repair these conditions, a surgeon typically inserts a vitrectomy probe into the posterior segment of the eye via a scleratomy, an incision through the sclera at the pars plana. The surgeon typically also inserts a fiber optic light source and an infusion cannula into the eye via similar incisions, and may sometimes substitute an aspiration probe for the vitrectomy probe. While viewing the posterior segment under a microscope and with the aid of the fiber optic light source, the surgeon cuts and aspirates away vitreous using the vitrectomy probe to gain access to the retinal detachment or tear. The surgeon may also use the vitrectomy probe, scissors, a pick, and/or forceps to remove any membrane that has contributed to the retinal detachment or tear. During this portion of the surgery, a saline solution is typically infused into the eye via the infusion cannula to maintain the appropriate intraocular pressure.

Next, the surgeon must manipulate the detached or torn portion of the retina to flatten against the choroid in the proper location. A soft tip cannula, forceps, or pick is typically utilized for such manipulation. Many surgeons also inject perfluorocarbon liquid as a retinal tamponading fluid into the posterior segment of the eye while aspirating the saline solution in the posterior segment to help cause the detached or torn portion of the retina to flatten against the choroid in the proper location. This procedure is typically referred to as a “fluid/perfluorocarbon” exchange. Other surgeons inject air as a retinal tamponading fluid into the posterior segment of the eye while aspirating the saline solution. This procedure is typically referred to as a “fluid/air” exchange. Finally, other surgeons inject a mixture of air and a gas such as SF₆, C₃F₈, or C₂F₆ as a retinal tamponading fluid into the posterior segment of the eye while aspirating the saline solution. This procedure is typically referred to as a “fluid/gas” exchange. As used herein, a “fluid” may include any liquid or gas that is suitable for use in the eye, including, but not limited to, saline solution with or without additives, silicone oil, a perfluorocarbon liquid, air, or a perfluorocarbon gas. The fluid exchange process is most typically performed by using a syringe filled with gas.

The process of filling the syringe with gas is currently time consuming. The process of filling the syringe with gas is a two person activity, requiring one sterile and one non-sterile person. Often times, the coordination of activity between the two individuals results in the loss of gas and a waste of time, and, possibly, the violation of the sterile field.

As a result, a need still exists in vitreoretinal surgery for an improved consumable for use in a fluid/gas exchange. The consumable should allow a scrub nurse to fill a syringe single handed, allow the nurse to maintain the integrity of the sterile field, eliminate the waste of expensive gas, and eliminate time lost as a result of mistakes.

### SUMMARY OF THE INVENTION

The present invention comprises a consumable for use in a fluid/gas exchange in vitreoretinal surgery. The consumable includes a syringe having a barrel with a first opening for receiving a retinal tamponading gas, a stopper slidably disposed within and fluidly sealed to the barrel, a plug assembly, and a plunger. The stopper has a distal cavity with a relief valve disposed therein. The plug assembly has a body for fluidly sealing an end of the barrel opposite the first opening, and a port fluidly coupled to an interior of the barrel and for fluidly coupling to a line containing pressurized air. The plunger has a handle for use by a user to slide the stopper within the barrel. The plunger is coupled to the stopper and fluidly and slidably sealed to the body. The plunger also has a hollow bore for fluidly coupling to the distal cavity when the relief valve is in an open position. The handle has an opening to atmosphere fluidly coupled to the hollow bore.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a schematic view of a surgical system including an automatic gas filling module and an automatic gas filling consumable according to a preferred embodiment of the present invention;

FIG. 2 is a perspective view of a second, preferred embodiment of the syringe of the automatic gas filling consumable of FIG. 1;

FIG. 3 is a cross-sectional view of the syringe of FIG. 2 taken along line 2-2;

FIG. 4 is a cross-sectional view of the syringe of FIG. 2 taken along line 3-3;

FIG. 5 is a cross-sectional view of a third, preferred embodiment of the syringe of the automatic gas filling consumable of FIG. 1; and

FIG. 6 is an enlarged, fragmentary view of the stopper of the syringe of FIG. 5.

### DETAILED DESCRIPTION OF THE PREFERRED EMBODIMENTS

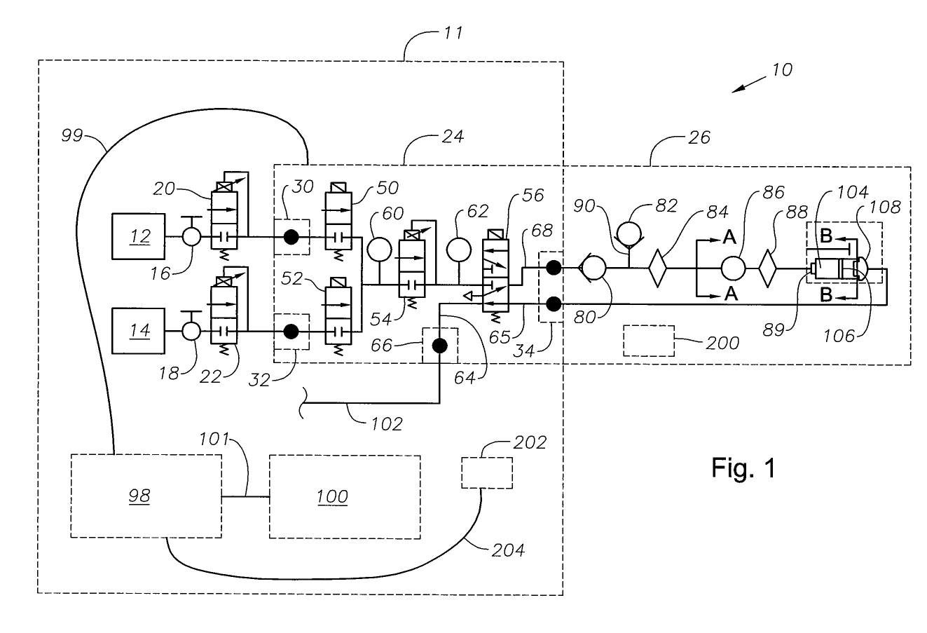

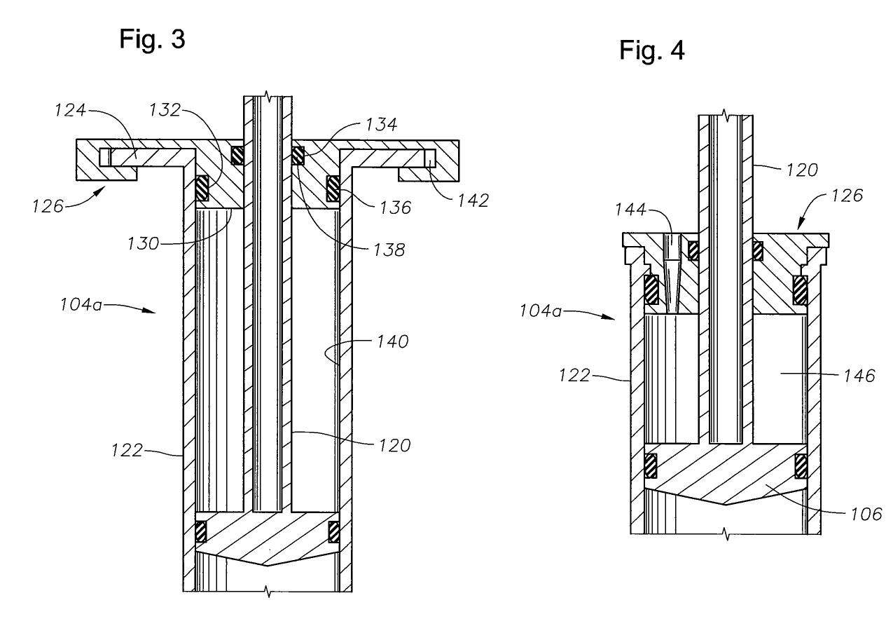

The preferred embodiments of the present invention and their advantages are best understood by referring to FIGS. 1-6 of the drawings, like numerals being used for like and corresponding parts of the various drawings.

Surgical system 10 generally includes a surgical console 11 and an automatic gas filling consumable 26. Surgical system 10 is preferably an ophthalmic surgical system.

Surgical console 11 preferably includes a pressurized gas bottle 12 having an integral valve 16 and regulator 20, a pressurized gas bottle 14 having an integral valve 18 and regulator 22, an automatic gas filling module 24 having an automatic gas filling port 34, a microprocessor 98 electrically coupled to automatic gas filling module 24 via an interface 99, a graphical user interface 100 electrically coupled to microprocessor 98 via interface 101, and a pressurized air line 102 capable of providing pressurized air in a proportional manner. Pressurized gas bottle 12 preferably holds a first retinal tamponading gas such as, by way of example, C₃F₈. Pressurized gas bottle 14 preferably holds a second retinal tamponading gas such as, by way of example, SF₆. Gas bottles 12 and 14, valves 16 and 18, and regulators 20 and 22 are fluidly coupled with automatic gas filling module 24 via connection points 30 and 32. Likewise, automatic gas filling module 24 is fluidly coupled with automatic gas filling consumable 26 via automatic gas filling port 34.

Automatic gas filling module 24 preferably includes shutoff valves 50 and 52, each of which is fluidly coupled with a regulator 54. Regulator 54 is fluidly coupled to timing valve 56. A pair of pressure transducers 60 and 62 are positioned on either side of regulator 54 to monitor gas pressure and flow. Pressurized air line 102 is fluidly coupled to automatic gas filling module 24 via connection point 66, and is also fluidly coupled with timing valve 56 via a gas line 64. A gas line 68 fluidly couples timing valve 56 and automatic gas filling port 34. A gas line 65 fluidly couples gas line 64 and automatic gas filling port 34 via timing valve 56.

Automatic gas filling consumable 26 preferably includes a check valve 80 fluidly coupled to automatic gas filling port 34 via gas line 68. A relief valve 82 is fluidly coupled with gas line 68 via a gas line 90. Gas line 68 also fluidly couples filter 84, stop cock 86, filter 88, and a distal end or opening 89 of a syringe 104. Pressurized air line 102 is fluidly coupled to an end cap 108 of syringe 104 via gas lines 64 and 65.

Gas bottles 12 and 14 are installed in console 11 with valves 16 and 18 open, and with regulators 20 and 22 pre-set. During operation, a scrub nurse will insert a sterile automatic gas filling consumable 26 into automatic gas filling port 34 on automatic gas filling module 24. Preferably, an RFID tag 200 on consumable 26 will be read by an RFID receiver 202 within surgical console 11. RFID receiver 202 is electrically coupled to microprocessor 98 via an interface 204. Surgical console 11 will thus detect that consumable 26 is an automatic gas filling consumable, and will populate the graphical user interface 100 appropriately. Alternatively, population of graphical user interface 100 may be performed manually in the event that RFID is not available.

Using graphical user interface 100, the scrub nurse will then select the retinal tamponading gas to be used and initiate the automatic gas filling process. At this point, depending on the retinal tamponading gas selected, microprocessor 98 opens one of gas shutoff valves 50 or 52. Regulator 54 will regulate the gas to a preset pressure that will flow to timing valve 56. Pressure transducers 60 and 62 will be monitored to verify that sufficient gas pressure and flow are available. In the event that sufficient gas pressure and flow are not available, microprocessor 98 will signal the scrub nurse via graphical user interface 100 that the active gas bottle 12 or 14 needs to be replaced.

Next, timing valve 56 will be energized, and retinal tamponading gas will flow through automatic gas filling port 34 into automatic gas filling consumable 26, and into distal end 89 of syringe 104. Gas pressure will overcome the friction of a stopper 106 within syringe 104, and stopper 106 will travel toward end cap 108, filling syringe 104 with retinal tamponading gas. Pressurized air within pressurized air line 102 will be vented to atmosphere during this process.

Timing valve 56 will then be closed and pressurized air from pressurized air line 102 will be supplied to end cap 108 of syringe 104, overcoming the friction of stopper 106 and allowing retinal tamponading gas to flow through syringe 104, filter 88, stop cock 86, and filter 84. Relief valve 82 is overcome so that retinal tamponading gas is vented to atmosphere. Microprocessor 98 repeats this cycle of introducing gas to syringe 104, and purging gas from syringe 104, a sufficient number of times until the concentration of retinal tamponading gas within syringe 104 is at or near 100%.

The scrub nurse will then remove end cap 108 from syringe 104 and will install a plunger (not shown) into syringe 104. The plunger is typically threaded into stopper 106. The scrub nurse then closes stop cock 86 and disconnects consumable 26 from surgical console 11 at section A. Gas filled syringe 104 is then presented to the surgeon for final mixing and administration. The portion of automatic gas filling consumable 26 that remains on console 11 will be removed and discarded when the case is complete.

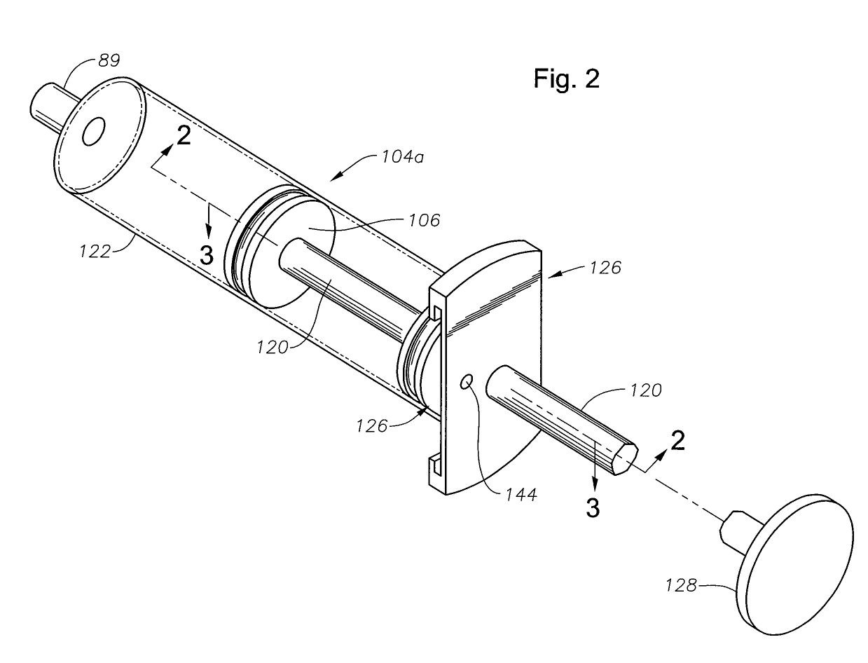

FIGS. 2-4 show a second, preferred embodiment of a syringe 104a of automatic gas filling consumable 26. Syringe 104a includes a plunger 120 coupled to stopper 106, a barrel 122 having a flange 124, and a plug assembly 126. Plunger 120 has a handle 128 on an end opposite of stopper 106.

Plug assembly 126 is sealingly coupled to barrel 122 and plunger 120. More specifically, plug assembly 126 includes a central body 130 disposed within barrel 122. Body 130 contains annular recesses 132 and 134 for retaining o-rings 136 and 138, respectively. O-ring 136 fluidly seals body 130 to an inside surface 140 of barrel 122. O-ring 134 fluidly and slidably seals body 130 to plunger 120. Plug assembly 126 also includes an annular recess 142 for receiving flange 124. Plug assembly 126 further includes a quick connect port 144 for fluidly and removably coupling with gas line 65. Port 144 is a luer type fitting or other port that allows gas line 65 to be quickly connected and disconnected to plug assembly 126. Port 144 is fluidly coupled to an interior 146 of barrel 122. As one skilled in the art may appreciate, syringe 104a allows a scrub nurse to prepare syringe 104a for use by a surgeon in a fluid/gas exchange without the steps of removing end cap 108 or threading a plunger into stopper 106. The time required for preparing the gas mixture for the fluid/gas exchange is thus reduced, and the possibility of losing or contaminating the plunger is eliminated.

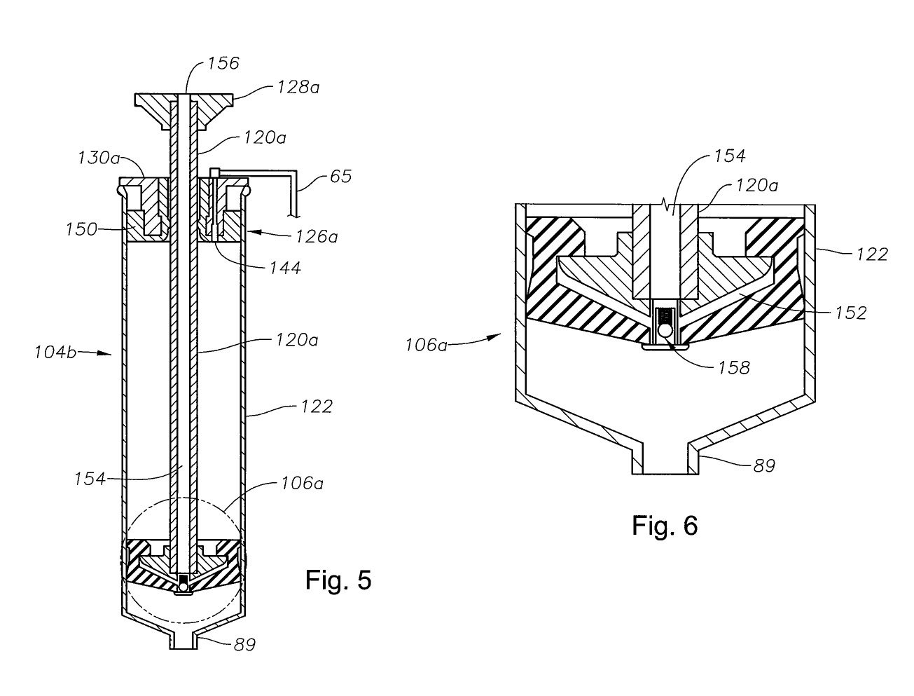

FIGS. 5-6 show a third, preferred embodiment of a syringe 104b of automatic gas filling consumable 26. Syringe 104b is substantially similar in structure to syringe 104a with the following exceptions. Syringe 104b includes a plug assembly 126a with a central body 130a and incorporated seals 150 that replace O-rings 136 and 138. Incorporated seals 150 may be made from an over-molded thermal plastic elastomer or a separately molded elastomer material that is assembled over central body 130a. Syringe 104b also includes a stopper 106a and a plunger 120a. Stopper 106a has a distal cavity 152. Plunger 120a has a hollow bore 154 terminating in an opening 156 in handle 128a. Stopper 106a has a relief valve 158 operatively disposed within cavity 152. When relief valve 158 is in an open position, cavity 152 and hollow bore 154 are fluidly coupled. When relief valve 158 is in its normally closed position, cavity 152 and hollow bore 154 are not fluidly coupled. The cracking pressure of relief valve 158 is slightly higher than the fill pressure of retinal tamponading gas set by regulator 54.

Syringe 104b permits relief valve 82, gas line 90, filter 84, and stopcock 86 to be eliminated from automatic gas filling consumable 26, providing a more simple and compact consumable design that is more easily managed by the scrub nurse. During operation, timing valve 56 is closed and pressurized air from pressurized air line 102 is preferably first supplied to port 144 of plug assembly 126a via gas line 65. The friction of stopper 106a is overcome, and stopper 106a moves toward distal end 89 of syringe 104b. As stopper 106a approaches distal end 89, relief valve 158 is overcome, and any air in front of stopper 106a is vented to atmosphere via hollow bore 154 and opening 156. Next, timing valve 56 is energized and retinal tamponading gas flows through automatic gas filling port 34, into automatic gas filling consumable 26, and into distal end 89 of syringe 104b. Regulator 54 sets the retinal tamponading gas fill pressure to slightly less than the cracking pressure of relief valve 158. Gas pressure overcomes the friction of stopper 106a, and stopper 106a travels toward plug assembly 126a, filling syringe 104b with retinal tamponading gas. Pressurized air within pressurized air line 102 is vented to atmosphere during this process. Timing valve 56 is then closed, and pressurized air from pressurized air line 102 is preferably supplied to port 144. Stopper 106a travels toward distal end 89. As stopper 106a approaches distal end 89, relief valve 158 is overcome and the retinal tamponading gas in front of stopper 106a is vented to atmosphere via hollow bore 154 and opening 156. Microprocessor 98 repeats this cycle of introducing gas to syringe 104b, and purging gas from syringe 104b, a sufficient number of times until the concentration of retinal tamponading gas within syringe 104b is at or near 100%.

From the above, it may be appreciated that the present invention provides an improved consumable for fluid/gas exchanges in vitreoretinal surgery. The consumable allows a scrub nurse to fill a gas syringe single handed, allows the nurse to maintain the integrity of the sterile field, eliminates the waste of expensive gas, and saves time lost due to mistakes.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A consumable for use in a fluid/gas exchange in vitreoretinal surgery, comprising:
a syringe having a barrel with a first opening for receiving a retinal tamponading gas;
a stopper slidably disposed within and fluidly sealed to said barrel, said stopper having a distal cavity with a relief valve disposed therein;
a plug assembly having:
a body for fluidly sealing an end of said barrel opposite said first opening; and
a port fluidly coupled to an interior of said barrel and for fluidly coupling to a line containing pressurized air; and
a plunger having a handle for use by a user to slide said stopper within said barrel, said plunger being coupled to said stopper and fluidly and slidably sealed to said body, said plunger having a hollow bore for fluidly coupling to said distal cavity when said relief valve is in an open position, said handle having an opening to atmosphere fluidly coupled to said hollow bore.
